(19) United States
(12) Patent Application Publication
(10) Pub. No.: US 2023/0149940 A1
(43) Pub. Date: May 18, 2023

(54) MASK DISPOSAL APPARATUS HAVING SERRATED TYPE CRUSHING STRUCTURE

(71) Applicant: Jun Young JOUNG, Cheongju-si (KR)

(72) Inventor: Jun Young JOUNG, Cheongju-si (KR)

(21) Appl. No.: 17/985,176

(22) Filed: Nov. 11, 2022

(30) Foreign Application Priority Data
Nov. 18, 2021 (KR) 10-2021-0159451

(51) Int. Cl. B02C 18/14; B02C 18/16; B02C 18/18; A61L 11/00

(52) U.S. Cl. CPC B02C 18/14; B02C 18/16; B02C 18/182; A61L 11/00

(57) ABSTRACT

Disclosed herein is a mask disposal apparatus having a serrated type crushing structure including: a main body including an inlet in which a wasted mask is put and a receiving space 110T disposed therein; and a crushing unit installed under an inlet in the main body to crush and discharge the wasted mask inserted into the inlet. The crushing unit includes: a rotary roller part provided in a cylindrical shape and having a first rotary shaft connected at the inner center thereof in a longitudinal direction; and a plurality of crushing parts having at least one serrated wheel portion having saw-teeth formed along the circumference thereof.

### BACKGROUND OF THE INVENTION

### Field of the Invention

The present invention relates to a mask disposal apparatus having a serrated type crushing structure.

### Background Art

Wastes which are likely to cause harm such as infection to the human body, and wastes which need special management for health and environmental protection, such as extractions from human tissues, dead bodies of experimental animals, or the likes, among wastes discharged from health care medical institutions, animal hospitals, test and inspection institutions, and the like, are defined as medical wastes. A disposal method capable of discharging the wastes in a container for medical wastes, storing them in a closed state, collecting and carrying them in a vehicle for medical wastes, and disposing them in an incineration facility or a sterilization facility for medical wastes is required.

In a case of masks among the medical wastes, since users' solution or secretion are stained on the masks, if the masks are simply sorted, collected and discharged, various kinds of germs or infections may occur during such a process.

Moreover, if the masks are discharged as general wastes after being used, they are not easily decomposed due to material characteristics of the textile material, and even if they are incinerated, it causes lots of environmental burdens. Therefore, people continuously demand solutions of the problems.

Recently, as an amount of masks used increases rapidly around the world, the amount of masks abandoned thoughtlessly is also increasing and the wasted masks flow into the marine environment, it has bad influences on water pollution and changes in the marine environment. Furthermore, since marine mammals mistake them for feed or an ear loop of a mask is caught to a body part of a marine mammal, it may cause a problem.

Therefore, it is necessary to develop an apparatus capable of effectively and easily disposing of used masks by completely separating various materials of the masks in safety in an aspect of disease prevention after the use of the masks.

In order to safely dispose of medical wastes, Korean Patent No. 10-1763381 discloses a “medical waste safe treatment apparatus having air purification function” (hereinafter, called ‘conventional art’).

However, the existing mask disposal apparatuses as well as the conventional art adopt a method of simply collecting, sterilizing and treating wasted masks. So, in a case of masks composed of various materials and having a many-fold filter structure, they cannot securely discard and treat the masks since being capable of treating only the exposed portions.

Furthermore, even if the mask disposal apparatuses according to the conventional arts adopt a method of crushing wasted masks, they cannot completely crush the masks having many-fold layer structure of fiber materials and just have a function to simply tear up the wasted masks. Accordingly, the conventional disposal apparatuses have a problem in that a crushed material is caught to a portion of the apparatus and it causes a breakdown or a function error of the apparatus.

### SUMMARY OF THE INVENTION

Accordingly, the present invention has been made to solve the above-mentioned problems occurring in the prior arts, and it is an object of the present invention to provide a mask disposal apparatus which can securely crush, separate, and break a mask having a many-fold filter layer structure composed of a fiber material, thereby improving a disposal level and preventing various problems caused by the mask which is not crushed properly and is caught to a portion of the apparatus.

It is another object of the present invention to provide a mask disposal apparatus which completely crushes a used mask, separates the filter layers, and breaks the mask to pieces, and sterilizes them, thereby preventing infection caused by bacteria or viruses remaining in the mask during collecting and incinerating processes of the mask.

To accomplish the above object, according to the present invention, there is provided a mask disposal apparatus having a serrated type crushing structure including: a main body including an inlet in which a wasted mask is put and a receiving space 110T disposed therein; and a crushing unit installed under an inlet in the main body to crush and discharge the wasted mask inserted into the inlet. The crushing unit includes: a rotary roller part provided in a cylindrical shape and having a first rotary shaft connected at the inner center thereof in a longitudinal direction; and a plurality of crushing parts having at least one serrated wheel portion having saw-teeth formed along the circumference thereof, and the plurality of crushing parts are rotatably installed on a second rotary shaft arranged to be parallel to the first rotary shaft of the rotary roller part on a plane, and a gap is formed between the crushing parts and the rotary roller part so that the wasted mask passing through the inlet is inserted into the gap to be crushed.

Here, the crushing part includes: a plurality of serrated wheel portions; and one or more tension wheel portions rotatably mounted on the second rotary shaft so as to be arranged between the neighboring serrated wheel portions, among the plurality of serrated wheel portions, in the longitudinal direction of the second rotary shaft, and having a cylindrical wheel structure made of an elastic material.

Moreover, the serrated wheel portion includes: a wheel body portion 123aa having has a cylindrical wheel structure and including a hollow rotary shaft insertion hole 123H, into which the second rotary shaft 123A is inserted, as a base structure; a first crushing blade portion having a sharply serrated crushing structure in which an end portion corresponding to the tip of the tooth is bent toward one side in a thickness direction of the wheel body portion; and a second crushing blade portion having a sharply serrated crushing structure in which an end portion corresponding to the tip of the tooth is bent toward the other side in the thickness direction of the wheel body portion.

Furthermore, the crushing unit further includes: a rotary motor part for rotating the first rotary shaft of the rotary roller part so that the wasted mask inserted into the inlet is pushed in a discharge direction when coming into contact with the outer circumferential surface of the rotary roller part.

Additionally, the crushing unit further includes an interval adjusting part for separating the neighboring crushing parts, among the plurality of crushing parts of which the inner center is rotatably mounted on the second rotary shaft, from each other at a predetermined interval in the longitudinal direction of the second rotary shaft.

In addition, the mask disposal apparatus further includes: a collection box disposed below the crushing unit located below the receiving space in the main body so that the pieces of the wasted mask crushed and discharged through the crushing unit fall down into a collection space disposed therein to be collected in the collection space; and a sterilization module mounted on one side of the receiving space in the main body and generating disinfectant materials for sterilizing the pieces of the wasted mask to provide the generated disinfectant materials to the collection space in the collection box.

According to the present invention, the following effects can be obtained.

First, the mask disposal apparatus according to the present invention can completely separate and crush a wasted mask of a many-fold filter layer structure made of a fiber material, and break the mask to pieces through the configuration, the arrangement form of the crushing unit and the structure of the crushing parts of the crushing unit.

Second, the mask disposal apparatus according to the present invention can completely remove an error in the driving of the entire device or in operation of some functions of the apparatus, caused when a mask which is not crushed completely and is torn partially is caught to a portion of the disposal apparatus.

Third, the mask disposal apparatus according to the present invention does not cause infection by bacteria or viruses remaining in the mask during the collection and incineration processes since performing a sterilization process after completely crushing and separating the wasted mask and breaking the mask to pieces.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view illustrating a mask disposal apparatus according to an embodiment of the present invention;

FIG. 2 is a side cross-sectional view illustrating an internal structure of the mask disposal apparatus according to the embodiment of the present invention;

FIG. 3 is a plan view illustrating a state in which an upper side of the mask disposal apparatus is open to confirm a structure of a crushing unit;

FIG. 4 is a view showing a configuration of a crushing part and components of the crushing unit in the mask disposal apparatus according to the embodiment of the present invention; and

FIG. 5 is a front view illustrating a serrated wheel portion of the crushing part of the crushing unit in the mask disposal apparatus according to the embodiment of the present invention.

### DETAILED DESCRIPTION OF THE PREFERRED EMBODIMENT

With reference to the accompanying drawings, preferred embodiments of the present invention will be described in more detail, wherein descriptions of known technical parts will be omitted or summarized for brief description.

### DESCRIPTION OF MASK DISPOSAL APPARATUS HAVING SERRATED TYPE CRUSHING STRUCTURE

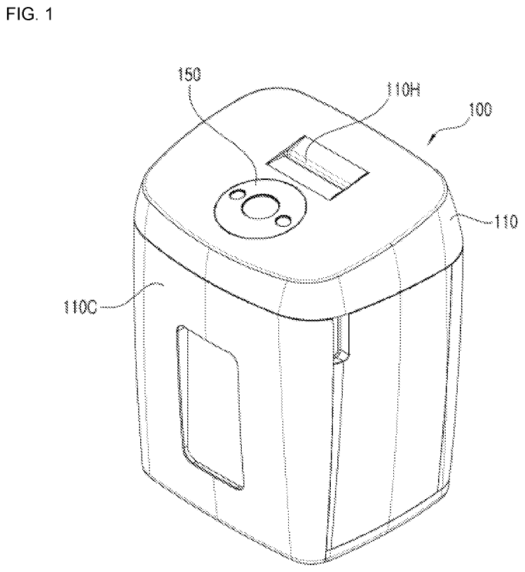

Referring to FIGS. 1 to 3, a mask disposal apparatus 100 having a serrated crushing structure according to an embodiment of the present invention includes a main body 110, a crushing unit 120, a sterilization module 130, a collection box 140, and an operation panel 150.

The main body 110 corresponds to an entire housing area and includes an inlet 110H disposed on an upper side thereof to put a wasted mask therein and a receiving space 110T disposed therein.

The crushing unit 120 is installed under an inlet in the main body 110, and includes a rotary roller part 121, a rotary motor part 122, a crushing part 123, and an interval adjusting part 124 in order to crush and discharge the wasted mask inserted into the inlet 110H.

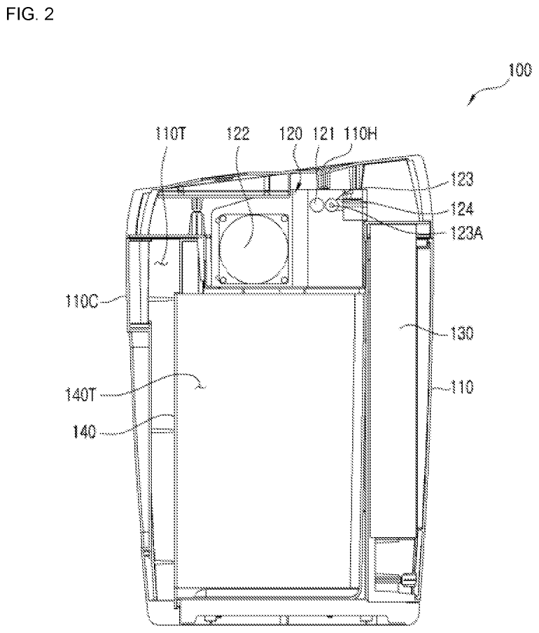

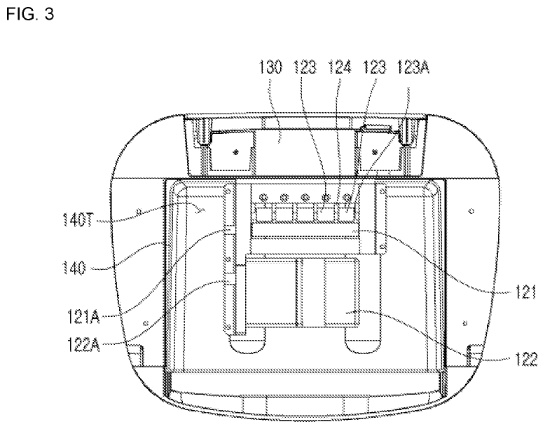

First, as illustrated in FIGS. 2 and 3, the rotary roller part 121 is provided in a cylindrical shape and is a roller means having a first rotary shaft 121A connected at the inner center thereof in a longitudinal direction.

The rotary motor part 122 rotates the first rotary shaft of the rotary roller part 121 to push the waste mask in a discharge direction (downward from the upper side) when the wasted mask inserted into the inlet 110H comes into contact with the outer circumferential surface of the rotary roller part 121.

For example, referring to FIG. 2, the rotary motor part 122 rotates the first rotary shaft 121A in the clockwise direction, and then, the rotary roller part 121 is also rotated in the clockwise direction, interlocking with the rotary motor part.

Here, the interlocking form between the rotary shaft 121A of the rotary motor part 122 and the first rotary shaft 121A may be made in various ways. For instance, the rotary shaft 121A of the rotary motor part 122 and the first rotary shaft 121A may be connected by one shaft or the first rotary shaft 121A may be rotated in the same direction as the rotary shaft 121A of the rotary motor part 122 by using a disc, a pulley, a belt, or the like.

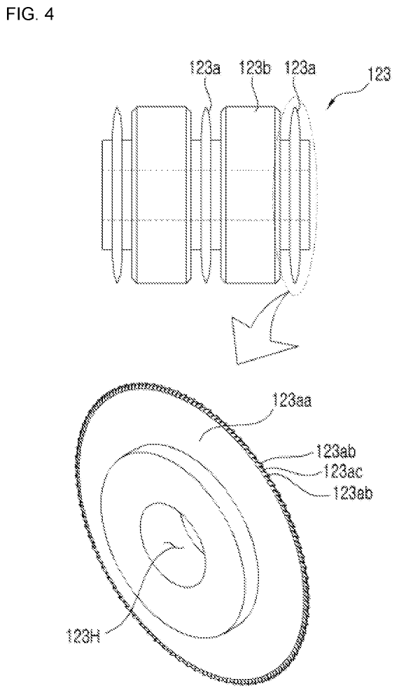

Next, as illustrated in FIGS. 3 and 4, the crushing part 123 includes at least one serrated wheel portion 123a having saw-teeth formed along the circumference thereof, and is provided in plurality.

In detail, the plurality of crushing parts 123 are rotatably installed on a second rotary shaft 123A arranged to be parallel to the first rotary shaft 121A of the rotary roller part 121 on a plane, and a gap is formed between the crushing parts 123 and the rotary roller part 121 so that the wasted mask passing through the inlet 110H is inserted into the gap to be crushed.

That is, the inlet 110H is formed in an elongated rectangular shape or in an oval shape, and the first rotary shaft 121A and the second rotary shaft 123A are arranged in parallel with each other on a plane in correspondence with the elongated direction of the inlet. The first rotary shaft 121A and the second rotary shaft 123A are mounted to be located at the same height.

Furthermore, the gam is formed between the outer circumferential surface of the rotary roller part 121 fixed and linked to the first rotary shaft 121A and the plurality of crushing parts 123 rotatably coupled to the second rotary shaft 123A in the longitudinal direction. The gap has a structure corresponding to the inlet 110H and is in a top and bottom arrangement, so that the wasted mask passing through the inlet 110H enters the gap formed between the outer circumferential surface of the rotary roller part 121 and the plurality of crushing parts 123 to be crushed as soon as falling down.

Here, the rotary roller part 121 which is fixed and coupled to the first rotary shaft 121A to interlock with the first rotary shaft is rotated by the rotary motor part 122 to downwardly pull and convey the wasted mask getting in contact therewith from the gap, and the plurality of crushing part 123 getting in contact with the other side completely crush the wasted mask to make it to pieces.

Here, the plurality of crushing parts 123 getting in contact with only one side surface of the wasted mask have a configuration of a special structure and a special arrangement form in order to perform a perfect crushing process.

In detail, as illustrated in FIG. 3, each unit of the crushing parts 123 includes a plurality of serrated wheel portions 123a having saw-teeth formed along the circumference thereof, and one or more tension wheel portions 123b of a cylindrical wheel structure made of an elastic material. The serrated wheel portions 123a and the tension wheel portions 123b have a shape in which the inner centers of serrated wheel portions 123a and the tension wheel portions 123b are rotatable by the second rotational shaft 123A.

Preferably, each unit of the crushing parts 123 has a structure in which the tension wheel portion 123b is disposed between the neighboring serrated wheel portions 123a among the plurality of serrated wheel portions 123a. The most preferably, each unit of the crushing parts 123 has a structure in which the serrated wheel portion 123a and the tension wheel portion 123b are arranged in order of ‘serrated wheel portion 123a-tension wheel portion 123b-serrated wheel portion 123a-tension wheel portion 123b-serrated wheel portion 123a in the longitudinal direction of the second rotary shaft 123A.

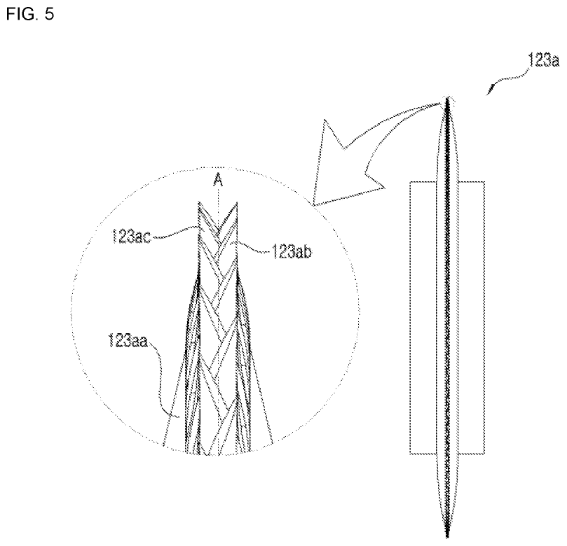

Here, as illustrated in FIGS. 4 and 5, the serrated wheel portion 123a includes: a wheel body portion 123aa having a cylindrical wheel structure and including a hollow rotary shaft insertion hole 123H, into which the second rotary shaft 123A is inserted, as a base structure; and a first crushing blade portion 123ab and a second crushing blade portion 123ac having different saw-teeth structures and alternately formed along the circumference thereof in the circumferential direction.

In addition, as illustrated in the enlarged portion of FIG. 5, the first crushing blade portion 123ab has a sharply serrated crushing structure in which an end portion corresponding to the tip of the tooth is bent toward one side (right side in FIG. 5) in a thickness direction (in the direction of the hollow rotary shaft insertion hole 123H, namely, in right-and-left direction of FIG. 5) of the wheel body portion 123aa.

Additionally, as illustrated in the enlarged portion of FIG. 5, the second crushing blade portion 123ac has a sharply serrated crushing structure in which an end portion corresponding to the tip of the tooth is bent toward the other side (left side in FIG. 5) in the thickness direction (in the direction of the hollow rotary shaft insertion hole 123H, namely, in right-and-left direction of FIG. 5) of the wheel body portion 123aa.

As a result, the plurality of serrated wheel portions 123a are alternately arranged in such a way that the sharp end portions of the serrated crushing structure are bent in the opposite direction, so that the surface of the mask getting in contact with the serrated wheel portions 123a can be completely crushed to pieces.

Next, the tension wheel portions 123b are arranged between the plurality of serrated wheel portion 123a as members of a cylindrical wheel structure made of an elastic material, such as a high-elastic sponge, thereby preventing crushed pieces of the mask from being jammed between the neighboring serrated wheel portions 123a, and applying tension to the contact surface through elasticity of a predetermined level so that the wasted mask which is being crushed is smoothly and stably pushed downwardly without being tangled by the rotary roller part 121.

The plurality of crushing parts 123 are not adjacently arranged on the second rotary shaft 123A in a lump, but are arranged to be spaced apart from one another at predetermined intervals by the interval adjusting part 124.

Here, the interval adjusting part 124 separates the neighboring crushing parts 123, among the plurality of crushing parts 123 of which the inner center is rotatably mounted on the second rotary shaft 123A, from each other at a predetermined interval in the longitudinal direction of the second rotary shaft 123A.

The crushing parts 123 spaced apart from one another by the interval adjusting part 124 and sequentially arranged on the second rotary shaft 123A have a sufficient distance between the serrated wheel portions 123a arranged at the outermost sides among the neighboring crushing parts 123 to prevent the crushed pieces of the mask from being jammed between the serrated wheel portions.

The sterilization module 130 is mounted on one side of the receiving space 110T in the main body 110, and generates disinfectant materials for sterilizing the pieces of the wasted mask to provide the disinfectant materials to a collection space 140T in the collection box 140.

In detail, the sterilization module 130 may be one selected among various sterilization devices, such as a plasma device for generating ozone and sterilizing air by irradiating extreme ultraviolet rays, a heating device for performing sterilization through temperature by applying predetermined heat, a sterilizer for killing pathogens with radicals and ions, and the like, and is not limited to such specific sterilization devices.

Ultimately, the sterilization module 130 generates sterilizing materials and supplies them to the inside of the collection box 140, so as to completely remove germs through sterilization after collecting the pieces of the wasted mask, thereby minimizing infectability during the disposal process of the pieces of the wasted mask collected in the collection box 140.

The collection box 140 is disposed below the crushing unit 120 located below the receiving space in the main body 110 so that the pieces of the wasted mask crushed and discharged through the crushing unit 120 fall down into the collection space 140T in the collection box 140 to be collected in the collection space.

As described above, the collection box 140 is mounted to be accommodated in the receiving space 110T or is removed from the receiving space 110T when a front cover 110C for covering the front side of the receiving space 110T is coupled to or uncoupled from the front of the main body 110.

The operation panel 150 is disposed on the upper surface of the main body 110 to generate a control signal by a user's pressing action, thereby controlling operational states of the rotary motor part 122 and the sterilization module 130.

The exemplary embodiments disclosed herein are not intended to limit the technical spirit but describe with the true scope and spirit being indicated by the following claims. The scope of the present invention should be construed based on the following appended claims and it should be construed that the technical spirit included within the scope equivalent to the claims belongs to the present invention.

What is claimed is:

1. A mask disposal apparatus having a serrated type crushing structure comprising:
a main body including an inlet in which a wasted mask is put and a receiving space 110T disposed therein; and
a crushing unit installed under an inlet in the main body to crush and discharge the wasted mask inserted into the inlet,
wherein the crushing unit comprises:
a rotary roller part provided in a cylindrical shape and having a first rotary shaft connected at the inner center thereof in a longitudinal direction; and
a plurality of crushing parts having at least one serrated wheel portion having saw-teeth formed along the circumference thereof, and
wherein the plurality of crushing parts are rotatably installed on a second rotary shaft arranged to be parallel to the first rotary shaft of the rotary roller part on a plane, and a gap is formed between the crushing parts and the rotary roller part so that the wasted mask passing through the inlet is inserted into the gap to be crushed.

2. The mask disposal apparatus according to claim 1, wherein the crushing part comprises:
a plurality of serrated wheel portions; and
one or more tension wheel portions rotatably mounted on the second rotary shaft so as to be arranged between the neighboring serrated wheel portions, among the plurality of serrated wheel portions, in the longitudinal direction of the second rotary shaft, and having a cylindrical wheel structure made of an elastic material.

3. The mask disposal apparatus according to claim 2, wherein the serrated wheel portion comprises:
a wheel body portion 123aa having has a cylindrical wheel structure and including a hollow rotary shaft insertion hole 123H, into which the second rotary shaft 123A is inserted, as a base structure;
a first crushing blade portion having a sharply serrated crushing structure in which an end portion corresponding to the tip of the tooth is bent toward one side in a thickness direction of the wheel body portion; and
a second crushing blade portion having a sharply serrated crushing structure in which an end portion corresponding to the tip of the tooth is bent toward the other side in the thickness direction of the wheel body portion.

4. The mask disposal apparatus according to claim 2, wherein the crushing unit further comprises:
a rotary motor part for rotating the first rotary shaft of the rotary roller part so that the wasted mask inserted into the inlet is pushed in a discharge direction when coming into contact with the outer circumferential surface of the rotary roller part.

5. The mask disposal apparatus according to claim 2, wherein the crushing unit further comprises:
an interval adjusting part for separating the neighboring crushing parts, among the plurality of crushing parts of which the inner center is rotatably mounted on the second rotary shaft, from each other at a predetermined interval in the longitudinal direction of the second rotary shaft.

6. The mask disposal apparatus according to claim 1, further comprising:
a collection box disposed below the crushing unit located below the receiving space in the main body so that the pieces of the wasted mask crushed and discharged through the crushing unit fall down into a collection space disposed therein to be collected in the collection space; and
a sterilization module mounted on one side of the receiving space in the main body and generating disinfectant materials for sterilizing the pieces of the wasted mask to provide the generated disinfectant materials to the collection space in the collection box.
